(19) United States
(12) Patent Application Publication
(10) Pub. No.: US 2022/0112439 A1
(43) Pub. Date: Apr. 14, 2022

(54) ESTOLIDE ESTERS AND USE THEREOF AS A BASE OIL IN LUBRICANTS

(71) Applicant: PETER GREVEN GmbH & Co. KG, Bad Munstereifel (DE)

(72) Inventors: Wilhelm HUBER, Kerpen (DE); Hermann Josef STOLZ, Bad Münstereifel (DE)

(21) Appl. No.: 17/424,383

(22) PCT Filed: Jan. 23, 2020
(86) PCT No.: PCT/EP2020/051661

§ 371 Date: Jul. 20, 2021

(30) Foreign Application Priority Data
Jan. 23, 2019 (EP) 19153186.2

(51) Int. Cl. C10M 105/42

(52) U.S. Cl. CPC C10M 105/42; C10N 2020/02; C10M 2207/301

(57) ABSTRACT

Estolide esters obtainable by esterification of—hydroxycarboxylic acids having 12 to 24 carbon atoms, the hydroxycarboxylic acids comprising unsaturated hydroxycarboxylic acids having a)—monocarboxylic acids with 6 to 22 carbon atoms and—polyols with at least two hydroxy groups or b)—monoalcohols having 8 to 22 carbon atoms and—linear carboxylic acids having at least two carboxyl groups or c)—monocarboxylic acids having 6 to 22 carbon atoms and—monoalcohols having 8 to 22 carbon atoms.

The present invention relates to estolide esters, the preparation thereof, and the use thereof as a base oil for lubricants, and to such lubricants.

Synthetic esters have been used for decades as base fluids or additives for lubricants. At first, the driving force of this development was concern about the finiteness of the crude oil resources. However, considerations relating to toxicity and biodegradability have increasingly come to play a critical role. The aspect of sustainability is becoming more and more important. Following the example of nature, closed material cycles of regenerable raw materials are to be utilized. An even CO₂ balance is one of the critical factors.

Lubricants are supposed to reduce friction between mobile surfaces. The wear between the workpieces is considerably reduced thereby, and excess heat development is prevented. In addition, the lubricants cool the surface and provide for an export of particles.

The different emphasis of these functions is substantially determined by the base fluids of the lubricants. Mineral oils are broadly employed. For the reasons mentioned above, they are increasingly replaced by ester.

Esters have a number of advantages over mineral oils. Because of the polarity of the ester group, they have a high affinity for metal surfaces. This results in good lubricant properties. They have a lower volatility than that of mineral oils, and a high viscosity index, which causes a lower degree of dependence on temperature. The viscosities of the esters can be varied in a broad range by esterification with dicarboxylic acids.

However, esters also have drawbacks. The oxidation stability and thermal stability is not always sufficient for critical applications, especially with some unsaturated esters. Alternatives on the basis of saturated fatty acids mostly show better values, but show worse results in long term cold performance, and often have an insufficient seal tolerance. In addition, the ester linkage is sensitive towards hydrolysis. This has disadvantageous effects, for example, in the production of fats. In many fats, metal soaps are used as thickeners. Lithium or calcium soaps are often employed. The in situ saponification of lithium hydroxide or calcium hydroxide or mixtures thereof with a suitable fatty acid in mineral oil is a common method. This is done at temperatures of more than 200° C. with the addition of water. If esters are used, this method cannot be applied.

Alternatively, the saponification is performed in a mineral oil, followed by filling up with ester oil, or else ready metal soaps are used. Other thickeners, such as polyurea, may also be used.

However, the desired results are often not obtained with these methods. There is still a need for alternative ester oils, which preferably overcome at least one of the mentioned drawbacks.

This object is achieved by estolide esters obtainable by the esterification of
hydroxycarboxylic acids with 12 to 24 carbon atoms, said hydroxycarboxylic acids including unsaturated hydroxycarboxylic acids, with
a)
   monocarboxylic acids with 6 to 22 carbon atoms; and
   polyols with at least two hydroxy groups;
or
b)
   monoalcohols with 8 to 22 carbon atoms; and
   linear carboxylic acids with at least two carboxy groups;
or
c)
   monocarboxylic acids with 6 to 22 carbon atoms; and
   monoalcohols with 8 to 22 carbon atoms.

Thus, the estolide esters according to the invention are obtainable by the esterification of hydroxyacids among themselves. This forms oligomeric or polymeric esters. In addition, either monocarboxylic acids or monoalcohols are contained in the reaction mixture. These serve as “capping agents”, preventing further oligomerization or polymerization.

Further, either polyols with at least two hydroxy groups or carboxylic acids with at least two carboxy groups are employed in embodiments a) and b). These serve for further cross-linking in order to increase the degree of oligomerization or polymerization. Such compounds are also referred to as compounding agents.

These are always employed in pairs, i.e., monocarboxylic acids as capping agents with polyols as compounding agents, or monoalcohols as capping agents with polyhydric acids as compounding agents.

In embodiment c), monocarboxylic acids with 6 to 22 carbon atoms are employed together with monoalcohols, thus serving as capping agents.

According to the invention, unsaturated hydroxycarboxylic acids are employed as hydroxycarboxylic acids. It is also possible to employ additional saturated hydroxycarboxylic acids.

Hydroxycarboxylic acids are carboxylic acids that bear at least one hydroxy group but may also bear more than one hydroxy groups.

Preferred lengths of the hydroxycarboxylic acids are 12 to 18 or 12 to 20 carbon atoms. They may be branched-chain or linear.

Preferred examples of such unsaturated hydroxycarboxylic acids include ricinoleic acid, lesquerolic acid, 15-hydroxylinoleic acid, auricolic acid, or hydroxypalmitoleic acid, and mixtures thereof. Ricinoleic acid is particularly preferred.

Preferably, the proportion of unsaturated hydroxycarboxylic acids in the hydroxycarboxylic acids is 10% by weight, more preferably at least 25% by weight, or at least 50% by weight, or at least 80% by weight, or at least 99% by weight.

In a preferred embodiment, the hydroxycarboxylic acids are virtually all unsaturated hydroxycarboxylic acids, within the limits of purity of technical hydroxycarboxylic acids.

As saturated fatty acids, for example, hydrogenated ricinoleic acid, hydroxypalmitic acid, or hydroxydodecanoic acid, or mixtures thereof may be contained.

In one embodiment, monocarboxylic acids with 6 to 22 carbon atoms, preferably from 6 to 18 carbon atoms, are employed as the second component.

Particularly suitable monocarboxylic acids include hexanoic acid, caprylic acid, caprinic acid, pelargonic acid, lauric acid, myristic acid, palmitic acid, stearic acid, oleic acid, linolic acid, linolenic acid, arachidonic acid, and behenic acid, or mixtures thereof. Further, isostearic acids and monomeric acids, in particular, are suitable branched monocarboxylic acids.

Monomer acids are by-products of the production of dimer fatty acids. Dimer fatty acids are produced from different fatty acids by decoction. A fatty acid with conjugated double bonds (conjuenic acid) and further unsaturated fatty acids are necessary. Examples of such fatty acids include conjugated linolic acid. The reaction occurs through a Diels-Alder addition, whereby a partially unsaturated C6 ring is formed. In addition to the dimer, trimers and monomers of the fatty acids may also be present in admixture. Monomer acids are separated from the reaction mixture by distillation.

Polyols are employed as a third component. Polyols are compounds that bear at least two hydroxy groups. As a delimitation from hydroxycarboxylic acids, they must not have a carboxylic acid function. Preferably, the polyols do not contain any β-hydrogen atoms.

Suitable examples of polyols include trimethylolpropane, di(trimethylol)propane, neopentyl glycol, pentaerythritol, dipentaerythritol, monoethylene glycol, (1,2-ethanediol), and mixtures thereof.

In a second embodiment, monoalcohols with 8 to 18 carbon atoms are employed as the second component.

Particular suitable monoalcohols include:
a) octanol, decanol, isotridecyl alcohol, and mixtures thereof;
b) mixtures of 2-alkyl-alcohols obtained by the Guerbet reaction, and mixtures thereof with a).

Polyhydric carboxylic acids, i.e., carboxylic acids with at least two carboxy groups, are then employed as the third component. As a delimitation from hydroxycarboxylic acids, they must not have any hydroxy functions.

Suitable polyhydric carboxylic acids include, in particular, 1,4-butanedioic acid (succinic acid), 1,6-hexanedioic acid (adipic acid), 1,9-nonanedioic acid (azelaic acid), and 1,10-decanedioic acid (sebacic acid), and mixtures thereof.

In some preferred embodiments, the monocarboxylic acids or monoalcohols (capping agents) are branched-chain, or at least there are branched-chain compounds contained in a mixture.

In some preferred embodiments, the carboxylic acids with at least two carboxy groups or polyols (compounding agents) are branched-chain, or at least there are branched-chain compounds contained in a mixture.

In some preferred embodiments, both compounding agents and capping agents are branched-chain, or at least there are branched-chain compounds contained in a mixture.

As the molar ratio of hydroxycarboxylic acids to monocarboxylic acids or monoalcohols (or to the sum of monocarboxylic acids or monoalcohols) in variant c)), a range of 3:1 to 1:1 has proven suitable.

As the molar ratio of hydroxycarboxylic acids to polyols, a ratio of 12:1 to 2:1, preferably 7:1 to 2:1, has proven suitable.

The estolide esters according to the invention typically have a viscosity of at least 80 mm²/s at 40° C. If monoalcohols and monoacids according to variant c) are exclusively used, the estolide esters according to the invention typically have a viscosity of at least 40 mm²/s at 40° C.

The invention also relates to a process for preparing the estolide esters according to the invention, comprising the steps of the esterification of
hydroxycarboxylic acids with 12 to 24 carbon atoms, said hydroxycarboxylic acids including unsaturated hydroxycarboxylic acids, with
a)
   monocarboxylic acids with 6 to 22 carbon atoms; and
   polyols with at least two hydroxy groups;
or
b)
   monoalcohols with 8 to 22 carbon atoms; and
   linear carboxylic acids with at least two carboxy groups;
or
c)
   monocarboxylic acids with 6 to 22 carbon atoms; and
   monoalcohols with 8 to 22 carbon atoms.

The invention further relates to the use of the estolide esters according to the invention as a base oil for lubricants, and to a lubricant comprising an estolide ester according to the invention.

Preferably, such lubricants comprise other additives selected from the group consisting of antioxidants, defoamers, extreme pressure additives, wear inhibitors, and pour point depressants. The invention is further illustrated by the following Examples.

### EXAMPLE 1

### Synthesis of an Estolide TMP Ester With a C8/C10 Fatty Acid (ISO-VG 150)

105.4 g of a C8/C10 fatty acid mixture (58:42% by weight) and 200 g of ricinoleic acid are charged in a three-necked flask with an intensive cooler and thermometer, and esterified at 180-250° C. under 100-250 mbar, until the hydroxyl number of the reaction mixture is <30 mg of KOH/g. The mixture is cooled down, and 30.0 g of 1,1,1-trimethylolpropane and 0.1 g of SnO as a catalyst are metered in. Subsequently, the product is esterified at 185-220° C. and under 100-250 mbar until the acid number is <2 mg of KOH/g. After removing the catalyst, the ester is obtained with a viscosity of about 150 mm²/s.

### EXAMPLE 2

### Synthesis of an Estolide NPG Ester With a C8/C10 Fatty Acid (ISO-VG 68)

105.8 g of a C8/C10 fatty acid mixture (58:42% by weight) and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 54.1 g of 2,2-dimethylolpropane and 0.1 g of SnO as a catalyst are metered in. Subsequently, the product is esterified at 185-220° C. and under 100-250 mbar until the acid number is <2 mg of KOH/g. After removing the catalyst, an ester is obtained with a viscosity of about 70 mm²/s.

### EXAMPLE 3

### Synthesis of an Estolide TMP Ester With a C8/C10 Fatty Acid (ISO-VG 320)

75.4 g of a C8/C10 fatty acid mixture (58:42% by weight) and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 28.5 g of 2,2-dimethylolpropane and 0.1 g of SnO as a catalyst are metered in. Subsequently, the product is esterified at 180-220° C. and under 10-100 mbar until the acid number is <2 mg of KOH/g. After removing the catalyst, the ester is obtained with a viscosity of about 320 mm²/s.

### EXAMPLE 4

### Synthesis of an Estolide ITD Ester With Adipic Acid (ISO-VG 100)

233.9 g of isotridecyl alcohol and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 62.5 g of adipic acid (1,6-hexanedioic acid) and 0.1 g of SnO as a catalyst are metered in. Subsequently, the product is esterified at 180-220° C. and under 10-100 mbar until the acid number is <2 mg of KOH/g. After removing the catalyst, an ester is obtained with a viscosity of about 100 mm²/s.

### EXAMPLE 5

### Synthesis of an Estolide MEG Ester With Isostearic Acid (ISO-VG 100)

399.7 g of isostearic acid and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 50.3 g of monoethylene glycol and 0.1 g of SnO as a catalyst are metered in. Subsequently, the product is esterified at 180-220° C. and under 10-100 mbar until the acid number is <2 mg of KOH/g. After removing the catalyst, an ester is obtained with a viscosity of about 100 mm²/s.

### EXAMPLE 6

### Synthesis of an Estolide ITD Ester With Isostearic Acid (ISO-VG 46)

399.7 g of isostearic acid and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 285.9 g of isotridecyl alcohol and 0.1 g of SnO as a catalyst are metered in. Subsequently, the product is esterified at 180-220° C. and under 10-100 mbar until the acid number is <2 mg of KOH/g. After removing the catalyst, an ester is obtained with a viscosity of about 46 mm²/s.

### EXAMPLE 7

### Synthesis of an Estolide TMP Ester With Isostearic Acid (ISO-VG 220)

399.7 g of isostearic acid and 333.1 g of ricinoleic acid are charged in a three-necked flask with an intensive cooler and thermometer, and esterified at 180-200° C. under 100-250 mbar, until the hydroxyl number of the reaction mixture is <30 mg of KOH/g. The mixture is cooled down, and 66.8 g of TMP and 0.1 g of SnO as a catalyst are metered in. Subsequently, the product is esterified at 180-220° C. and under 10-100 mbar until the acid number is <2 mg of KOH/g. After removing the catalyst, an ester is obtained with a viscosity of about 225 mm²/s.

Determination of the Hydrolytic Stability According to ASTM D2619

The determination of the hydrolytic stability of the estolides was effected according to ASTM D2619. The difference of the acid numbers before and after the test was used as the measured quantity. The values for the estolides according to Examples 1 to 3 were compared with complex esters of the respective ISO VG classes.

The values stated in Table 1 were obtained.

Determination of the Pour Point

The pour point of the estolides was determined in accordance with ISO 3016:1994. Estolides according to the invention and those of adipic and dimer fatty acids were compared. Here too, the values for the estolides according to Examples 1 to 3 were compared with complex esters of the respective ISO VG classes.

The values stated in Table 1 were obtained.

| | | | | Hydrolytic | |
|---|---|---|---|---|---|
| Viscosity class | | Carboxylic | | stability | Pour point |
| (ISO VG) | Polyol | acid | Fatty acids | [mg of KOH/g] | [° C.] |
| 150 (Example | TMP | caprylic/ | ricinoleic | 0.04 | −36 |
| 1) | | caprinic acid | acid | | |
| 150 (Comparative | TMP | adipic acid | oleic fatty | 0.2 | −30 |
| Example) | | | acid | | |
| 68 (Example | NPG | caprylic/ | ricinoleic | 0.05 | −51 |
| 2) | | caprinic acid | acid | | |
| 320 (Example | TMP | caprylic/ | ricinoleic | 0.22 | −36 |
| 3) | | caprinic acid | acid | | |
| 320 (Comparative | TMP | dimer fatty | oleic fatty | 0.21 | −30 |
| Example) | | acid | acid | | |
| 100 (Example | ITD | adipic acid | ricinoleic | 0.32 | −45 |
| 4) | | | acid | | |
| 100 (Example | MEG | isostearine | ricinoleic | 0.06 | −24 |
| 5) | | | acid | | |
| 46 (Example | ITD | isostearine | ricinoleic | 0.01 | −36 |
| 6) | | | acid | | |
| 220 (Example | TMP | isostearine | ricinoleic | 0.05 | −42 |
| 7) | | | acid | | |

1. An estolide esters obtainable by the esterification of
hydroxycarboxylic acids with 12 to 24 carbon atoms, said hydroxycarboxylic acids including unsaturated hydroxycarboxylic acids, with
a)
monocarboxylic acids with 6 to 22 carbon atoms; and
polyols with at least two hydroxy groups;
or
b)
monoalcohols with 8 to 22 carbon atoms; and
linear carboxylic acids with at least two carboxy groups;
or
c)
monocarboxylic acids with 6 to 22 carbon atoms; and
monoalcohols with 8 to 22 carbon atoms.

2. The estolide esters according to claim 1, wherein said unsaturated hydroxycarboxylic acids contain one or more double bonds.

3. The estolide esters according to claim 1, wherein said hydroxycarboxylic acids contain one or more hydroxy groups.

4. The estolide esters according to claim 1, wherein said monocarboxylic acids or said carboxylic acids with at least two carboxy groups are saturated or unsaturated monocarboxylic acids.

5. The estolide esters according to claim 1, wherein said hydroxycarboxylic acids have 12 to 18 carbon atoms.

6. The estolide esters according to claim 1, wherein the molar ratio of hydroxycarboxylic acids to polyols or carboxylic acids with at least two carboxy groups is within a range of from 12:1 to 2:1.

7. The estolide esters according to claim 1, wherein the molar ratio of hydroxycarboxylic acids to monocarboxylic acids or monoalcohols is within a range of from 3:1 to 1:1.

8. The estolide esters according to claim 1, wherein said polyols are polyhydric alcohols without any β-hydrogen atoms.

9. The estolide esters according to claim 1, wherein said polyols are selected from trimethylolpropane, di(trimethylol)propane, neopentyl glycol, pentaerythritol, dipentaerythritol, isotridecanol, 2-alkyl-alcohols (Guerbet alcohols), and mixtures thereof.

10. The estolide esters according to claim 1, wherein said unsaturated hydroxycarboxylic acids are selected from ricinoleic acid, lesquerolic acid, 15-hydroxylinoleic acid, auricolic acid, or hydroxypalmitoleic acid, or mixtures thereof.

11. The estolide esters according to claim 1, wherein said monocarboxylic acids are selected from hexanoic acid, caprylic acid, caprinic acid, pelargonic acid, lauric acid, myristic acid, palmitic acid, stearic acid, oleic acid, linolic acid, linolenic acid, arachidonic acid, behenic acid, isostearic acids, monomer acids, and mixtures thereof.

12. The estolide esters according to claim 1, wherein said carboxylic acids with at least two carboxy groups are selected from 1,4-butanedioic acid (succinic acid), 1,6-hexanedioic acid (adipic acid), 1,9-nonanedioic acid (azelaic acid), and 1,10-decanedioic acid (sebacic acid), and mixtures thereof.

13. The estolide esters according to claim 1, wherein said monoalcohols are selected from octanol, decanol, isotridecyl alcohol, 2-alkyl-alcohols obtained by the Guerbet reaction, and mixtures thereof.

14. The estolide esters according to claim 1, wherein the viscosity is at least 40 mm²/s at 40° C., or at least 100 mm²/s at 25° C.

15. A process for preparing estolide esters according to claim 1, comprising the steps of performing the esterification of
hydroxycarboxylic acids with 12 to 24 carbon atoms, said hydroxycarboxylic acids including unsaturated hydroxycarboxylic acids, with
a)
monocarboxylic acids with 6 to 22 carbon atoms; and
polyols with at least two hydroxy groups;
or
b)
monoalcohols with 8 to 22 carbon atoms; and
linear carboxylic acids with at least two carboxy groups;
or
c)
monocarboxylic acids with 6 to 22 carbon atoms; and
monoalcohols with 8 to 22 carbon atoms.

16. (canceled)

17. A lubricant comprising an estolide ester according to claim 1.
